(19) United States
(12) Patent Application Publication
(10) Pub. No.: US 2016/0237616 A1
(43) Pub. Date: Aug. 18, 2016

(54) METHOD FOR TREATING CELLULOSE-CONTAINING BIOMASS

(71) Applicant: SHOWA DENKO K.K., Tokyo (JP)

(72) Inventors: Ichiro FUJITA, Tokyo (JP); Shinji YAMAKI, Tokyo (JP); Tadashi YONEDA, Tokyo (JP)

(73) Assignee: SHOWA DENKO K.K., Tokyo (JP)

(21) Appl. No.: 15/027,550

(22) PCT Filed: Sep. 9, 2014
(86) PCT No.: PCT/JP2014/073794

§ 371 Date: Apr. 6, 2016

(30) Foreign Application Priority Data
Oct. 7, 2013 (JP) 2013-210210

(51) Int. Cl. D21B 1/06; B02C 19/00; C12P 19/14; B30B 11/24; C13K 1/02; C12P 19/02; B02C 19/22; B30B 15/34

(52) U.S. Cl. CPC D21B 1/063; B02C 19/22; B02C 19/0056; B30B 15/34; B30B 11/24; C13K 1/02; C12P 19/02; C12P 19/14; C12P 2201/00

(57) ABSTRACT

A treatment method for biomass, which is a pretreatment method including continuously performing hydrothermal treatment using a screw extruder to produce a biomass composition for saccharification from cellulose-containing biomass serving as a raw material, the treatment method sequentially including: pulverizing the cellulose-containing biomass serving as a raw material so as to have a maximum grain size of 1,000 μm or less and adjusting a water content ratio thereof to from 20% to 80%; performing, in a heating section of the screw extruder, hydrothermal treatment at a temperature of from 150° C. to 250° C. for 0.1 minute to 10 minutes while performing kneading pulverization having a grinding effect; and cooling, in a cooling section of the screw extruder downstream of the heating section, a treated product to 100° C. or less to recover the treated product of the cellulose-containing biomass having an average grain size of 100 μm or less.

### TECHNICAL FIELD

The present invention relates to a treatment method for cellulose-containing biomass. More specifically, the present invention relates to a treatment method for cellulose-containing biomass involving continuously performing hydrothermal treatment through use of a screw extruder to produce a biomass composition exhibiting high saccharification performance from the cellulose-containing biomass serving as a raw material, a production method for a biomass composition for saccharification, and a production method for a sugar.

### BACKGROUND ART

As part of measures against global warming, there have been wide investigations on production of various chemical products including ethanol and the like through effective utilization of cellulose-containing biomass. Examples of the cellulose-containing biomass include hard biomass such as cedar or cypress, and soft biomass, such as rice straw, wheat straw, corncobs, cassava, bagasse, or sugar cane leaves. The biomass may contain hemicellulose, lignin, and the like, and hence it is difficult to directly saccharify the biomass. Therefore, there have been proposals to enhance its saccharification performance through various pretreatments.

As a pretreatment method for enhancing the saccharification performance, there have been proposed a method involving adding an acid or alkali and performing hydrothermal treatment, and a method involving a combination of hydrothermal treatment without a chemical and physical pulverization treatment (JP 2006-136263 A; Patent Document 1). Further, in addition to those methods, there have been proposed water vapor blasting, ammonia blasting, ozone oxidation, white-rot fungus treatment, microwave irradiation, electron beam irradiation, and y-ray irradiation (Journal of The Japan Wood Research Society, 53, 1-13 (2007); Non-Patent Document 1).

However, when those pretreatment methods are studied for industrially useful treatment steps, a device and a method capable of continuously and efficiently treating a raw material in a large amount are not specifically disclosed, while those methods each have an effect of enhancing the saccharification performance to some extent.

As a method of continuously and efficiently performing pretreatment prior to enzymatic saccharification of biomass, in JP 59-192093 A (Patent Document 2), and JP 59-192094 A and JP 2012-170355 A (Patent Documents 3 and 4; U.S. Pat. No. 4,642,287), it has been proposed that a pretreatment method involving kneading biomass with an alkali and subjecting the biomass to hydrothermal treatment with a twin-screw extruder can be continuously performed at a high concentration in a short treatment time as compared to conventional fine pulverization treatment and alkali steam treatment.

However, the pretreatment methods disclosed in Patent Documents 2 to 4 each involve high chemical cost owing to the use of the alkali at a ratio of around 20% with respect to a raw material, and inevitably require neutralization and washing of the added alkali prior to the enzymatic saccharification. Therefore, a problem in terms of economy and efficiency including even a saccharification step is not solved. Further, the pretreatment methods of Patent Documents 2 and 3 are each disclosed as substantially a combination of pulverization and alkali steaming, but with regard to the treatment conditions, only the conditions related to the alkali steaming, such as a heating temperature, a heating time, and the amounts of the raw material and the alkali to be loaded, are presented, and the configuration of a device related to the pulverization, etc. are not presented. The embodiments for carrying out those inventions are unclear.

As a method of easily and rapidly performing pretreatment of plant biomass, in JP 2011-130745 A (Patent Document 5), there has been proposed a method involving sequentially performing pretreatment operations in an extruder in a continuous manner, the operations involving adding a decomposer to plant biomass coarsely pulverized into a preset size or less and subjecting the plant biomass to pressurized hot water treatment, and subsequent operations before saccharification loading in which the plant biomass is mixed with an enzyme for saccharification. However, in Patent Document 5, the conditions under which the treatment method is performed, and data on saccharification of the biomass as to what level of saccharification performance is obtained are not presented, while a flowchart and a screw configuration of the extruder for the method are disclosed. The overall performance and efficiency including even a saccharification step are unclear.

In view of the foregoing, there is a demand for establishment of a pretreatment method for cellulose-containing biomass which is high not only in saccharification performance of a treated product but also in overall production efficiency including even sugar production, capable of continuously treating a raw material in a large amount, and industrially useful.

### PRIOR ART

### Patent Document

Patent Document 1: JP 2006-136263 A

Patent Document 2: JP 59-192093 A (U.S. Pat. No. 4,642,287)

Patent Document 3: JP 59-192094 A (U.S. Pat. No. 4,642,287)

Patent Document 4: JP 2012-170355 A

Patent Document 5: JP 2011-130745 A

### Non-Patent Document

Non-Patent Document 1: Journal of the Japan Wood Research Society, 53, 1-13 (2007)

### DISCLOSURE OF INVENTION

### Problem to be Solved by Invention

An object of the present invention is to provide a treatment method for cellulose-containing biomass which is capable of continuously providing a cellulose-containing composition exhibiting high saccharification performance to glucose, and is industrially highly useful, a production method for a cellulose-containing composition for saccharification including conducting the treatment method, and a production method for a sugar including hydrolyzing the cellulose-containing composition for saccharification.

### Means to Solve Problem

The inventors of the present invention have made extensive investigations in order to achieve the above-mentioned object. As a result, the inventors have found that, when, in a treatment method for obtaining a sugar from cellulose-containing biomass, a screw extruder is used, and the cellulose-containing biomass is sequentially subjected to:

pulverization and adjustment of a water content ratio in a pulverization section of the device; hydrothermal treatment concurrently with kneading pulverization having a grinding effect in a heating section of the device; and cooling in a cooling section of the device, in a continuous manner, a cellulose-containing composition exhibiting high saccharification performance to glucose is obtained. Thus, the present invention has been completed.

That is, the present invention relates to the following treatment method for cellulose-containing biomass.
[1] A treatment method for biomass, which is a pretreatment method comprising continuously performing hydrothermal treatment through use of a screw extruder to produce a biomass composition for saccharification from cellulose-containing biomass serving as a raw material, the treatment method sequentially comprising: pulverizing, in a pulverization section of the screw extruder, the cellulose-containing biomass serving as a raw material so as to have a maximum grain size of 1,000 μm or less and adjusting a water content ratio thereof to from 20% to 80%; performing, in a heating section of the screw extruder, hydrothermal treatment at a temperature of from 150° C. to 250° C. for 0.1 minute to 10 minutes while performing kneading pulverization having a grinding effect; and cooling, in a cooling section of the screw extruder downstream of the heating section, a treated product to 100° C. or less to recover the treated product of the cellulose-containing biomass having an average grain size of 100 μm or less.
[2] The treatment method for biomass according to [1] above, in which, in the heating section, the kneading pulverization having a grinding effect is performed concurrently with the hydrothermal treatment by arranging an element including a seal ring and at least one set of a kneading disc and/or a left-hand screw arranged immediately upstream of the seal ring.
[3] The treatment method for biomass according to [1] or [2] above, in which, in the cooling section downstream of the heating section, the cooling is performed by installing a water cooling jacket and/or a liquid feed line.
[4] The treatment method for biomass according to any one of [1] to [3] above, in which the screw extruder is a co-rotating twin-screw extruder.
[5] The treatment method for biomass according to any one of [1] to [4] above, in which the cellulose-containing biomass is soft biomass.
[6] A production method for a biomass composition for saccharification, comprising conducting the treatment method described in any one of [1] to [5] above.
[7] A production method for a sugar, comprising hydrolyzing a biomass composition obtained by the production method described in [6] above.

### Effects of Invention

According to the treatment method for cellulose-containing biomass of the present invention comprising continuously performing the hydrothermal treatment concurrently with the kneading pulverization having a grinding effect through use of a screw extruder, a cellulose-containing composition for saccharification useful as a raw material for producing a sugar through a hydrolysis reaction is obtained, and a sugar can be efficiently produced from the cellulose-containing biomass.

### BRIEF DESCRIPTION OF DRAWINGS

FIG. 1(A) is a configuration view of a screw extruder A used in Examples 3, 5, 6 and 7, FIG. 1(B) is a configuration view of a screw extruder B used in Example 1, FIG. 1(C) is a configuration view of a screw extruder C used in Examples 2, 4, 8 and 9, FIG. 1(D) is a configuration view of a screw extruder D used in Comparative Example 28, and FIG. 1(E) is a configuration view of a screw extruder E used in Comparative Example 27.

FIG. 2 includes graphs for showing a relationship between a hydrothermal time and a saccharification rate in Examples 2 to 7, and 9, and Comparative Examples 1 to 17.

FIG. 3 includes graphs for showing a relationship between a solid content concentration at the time of hydrothermal treatment and a saccharification rate in Examples 3 to 5 and Comparative Examples 4, 7, 11, and 18 to 26.

### MODE FOR CARRYING OUT INVENTION

The present invention is described in detail below. A treatment method of the present invention comprises feeding cellulose-containing biomass to a screw extruder, and continuously performing hydrothermal treatment concurrently with kneading pulverization having a grinding effect. An acid or alkali as an additive may be added to water in a raw material used in the treatment method, but it is industrially preferred to use only water, which is generally available, because the use of the additive not only increases chemical cost but also produces cost for detoxification, such as neutralization, in a subsequent step.

[Cellulose-containing Biomass]

The biomass used in the treatment method of the present invention means a biopolymer (nucleic acid, protein, or polysaccharide) or an industrial resource derived from such constituent component, other than exhaustible resources (fossil fuel, such as petroleum, coal, or natural gas). Therefore, examples of the cellulose-containing biomass include hard biomass such as wood, and soft biomass such as rice straw, wheat straw, corncobs, cassava, bagasse, or sugar cane leaves. Soft biomass is preferred in consideration of the ease of the pretreatment, and further, bagasse and sugar cane leaves are particularly preferred in consideration of their global storage potential and collection cost.

[Type of Screw Extruder]

The screw extruder to be used in the treatment method of the present invention may be any one of a single-screw extruder, a multi-screw extruder, and a special extruder. Of those, a multi-screw extruder, which can apply stronger shear to a biomass material, is preferred, and a twin-screw extruder is more preferred because of its generality and versatility.

As the multi-screw extruder, there may be adopted any one of a type in which screw shafts are parallel to one another and a conical type in which the screw shafts cross obliquely one another. Of those, a parallel type is preferred. Any one of an engaged-screw type and a non-engaged-screw type may be adopted, but of those, an engaged-screw type is preferred because of a high kneading effect and many practical examples.

With regard to a screw rotation direction, any one of a co-rotation type and a counter-rotation type may be adopted, but of those, a co-rotation type is preferred because of a self-cleaning effect.

[Raw Material Feed Portion of Screw Extruder]

A hopper to be used for a raw material feed port configured to stably feed a raw material to a cylinder of the screw extruder is not limited as long as the hopper has a function capable of generating a feed pressure required for a feed portion of a screw without causing bridging, and examples of such hopper include a vibration hopper, a hopper with a force feeder, a hopper dryer, a vacuum hopper, and a nitrogen purge hopper. A hopper comprising a screw located on the inside of the hopper and configured to forcibly push a material into the cylinder is preferred from the viewpoint of stably feeding the raw material.

A device configured to quantitatively feed the raw material to the screw extruder is mounted below the hopper. The quantitative feeding device is not limited as long as the device has a function of enabling quantitative feeding, and examples of such device include a mass feeder and a constant volume feeder. Of those, a mass feeder is preferred in view of feeding raw material biomass, which generally has a low bulk density and non-uniform shapes and sizes. In order to feed the raw material to the screw extruder more securely, it is preferred to mount a compactor configured to forcibly press the raw material into the extruder through use of a screw or a piston so that the bulk density of the material can be increased.

[Cylinder Portion of Screw Extruder]

A cylinder portion of the screw extruder includes the following three sections: a heating section, which is located in the middle portion of the cylinder, and is configured to perform hydrothermal treatment through heating with a heater while grinding the raw material; a pulverization section, which is located upstream of the heating section, and is configured to pulverize the raw material and adjust its water content ratio, to thereby consolidate the material and maintain airtightness; and a cooling section, which is located downstream of the heating section, and is configured to cool the material, to thereby consolidate the material and maintain the airtightness. The screw extruder has an L/D of preferably from 30 to 80, more preferably from 40 to 80, still more preferably from 50 to 80 in its entirety including the pulverization section, the heating section, and the cooling section from the viewpoint of stably maintaining sealing, and performing hydrothermal treatment having an effect of improving the saccharification performance of the raw material biomass. The pulverization section has an L/D of preferably from 10 to 40, more preferably from 10 to 30, still more preferably from 15 to 25. In addition, the heating section has an L/D of preferably from 10 to 65, more preferably from 15 to 60, still more preferably from 20 to 55. The cooling section has an L/D of preferably from 5 to 35, more preferably from 5 to 20, still more preferably from 5 to 10. It should be noted that the “L/D” refers to an effective length represented by a ratio between the length (L) of a screw, which is measured from a start point of screw thread below the hopper to the tip of the screw, and the diameter (D) of the screw.

[Configuration of Pulverization Section of Cylinder]

The pulverization section of the cylinder preferably has a screw configuration in which at least one or more elements each comprising a seal ring and at least one set of a kneading disc (feed kneading disc, neutral kneading disc, or reverse kneading disc) or a left-hand screw arranged upstream of the seal ring (hereinafter abbreviated as “seal ring elements”) are arranged. A state in which the raw material on an upstream side is compressed is achieved by a damming effect exhibited by the arranged seal ring, thereby achieving a state in which the shear force of a screw located upstream of the seal ring is increased. As a result, the raw material is efficiently pulverized and consolidated, and exhibits a function of sealing the pressure of vapor to be generated in the heating section. The pulverization of the raw material is performed not only for achieving the sealing function but also for improving efficiency of the hydrothermal treatment in the heating section. For this, the maximum grain size of the raw material is preferably set to 1,000 μm or less. It should be noted that the maximum grain size is determined through microscopic observation of a sample extracted from the pulverization section immediately upstream of the heating section.

In addition, in the pulverization section, the water content ratio of the raw material is adjusted to preferably from 30 mass % to 80 mass %, more preferably from 30 mass % to 75 mass %, still more preferably from 35 mass % to 70 mass % in order to optimally perform a hydrothermal reaction and achieve an optimal sealing property. The adjustment of the water content ratio may be separately performed before loading, but is preferably performed by installing a liquid feed line in an arbitrary portion of the pulverization section to feed water therethrough with a high-pressure pump from the viewpoint of reducing the number of steps. It should be noted that the water content ratio refers to the ratio of the mass of water to the total mass of the raw material as it is.

[Configuration of Heating Section of Cylinder]

The heating section of the cylinder preferably has a screw configuration in which at least three or more sets of seal ring elements are arranged. The arrangement of a plurality of seal rings in the heating section exhibits such effect that strong grinding stress, which is generated when the cellulose-containing biomass serving as a raw material passes through an extremely narrow clearance between the seal ring and the cylinder, is applied concurrently with the hydrothermal treatment, thereby improving the saccharification performance of cellulose in biomass. For such effect, the clearance between the seal ring and the cylinder is preferably from 0.5% to 10.0%, more preferably from 1.0% to 8.0%, still more preferably from 1.5% to 5.0% with respect to the inner diameter of the cylinder. It should be noted that the inner diameter of the cylinder in a twin-screw extruder refers to the diameter of a circle surrounding one screw in a vertical cross section of the cylinder. The heating in the heating section is not limited as long as the cylinder can be heated, but is preferably performed with an electric heater from the viewpoint of temperature controllability. As the conditions of the hydrothermal treatment for the raw material, the temperature of the raw material falls within a range of preferably from 150° C. to 250° C., more preferably from 160° C. to 240° C., still more preferably from 170° C. to 230° C. A time of passage through the heating section falls within a range of preferably from 0.1 minute to 10 minutes, more preferably from 0.2 minute to 7.5 minutes, still more preferably from 0.3 minute to 7.5 minutes. A pressure in the heating section falls within a range of preferably from 0.1 MPa to 20 MPa, more preferably from 1 MPa to 15 MPa, still more preferably from 2 MPa to 12 MPa.

[Configuration of Cooling Section of Cylinder]

The cooling section of the cylinder preferably includes a water cooling jacket and/or a liquid feed line in order to cool the raw material heated in the heating section. The cooling in the cooling section is performed so that the temperature of the raw material is reduced to preferably 100° C. or less, more preferably 80° C. or less, still more preferably 70° C. or less. With this, vapor generated in the heating section turns into water, and the pressure of vapor flowing downstream together with the treated biomass can be sealed. Further, a pressure regulating valve may be mounted to a discharge port in the cooling section in order to seal the pressure of vapor more stably in the system.

[Pulverization (Adjustment of Grain Size)]

In the treatment method of the present invention, the cellulose-containing biomass serving as a raw material may be directly fed to the screw extruder without pulverization treatment, but is preferably subjected to adjustment of a grain size in advance through coarse pulverization before its feeding. Pulverization means is not particularly limited as long as the means has a function capable of pulverizing a solid substance. For example, the mode of the device may be a dry mode or a wet mode. In addition, the pulverization system of the device may be a batch system or a continuous system. Further, the pulverization force of the device may be provided by any of impact, compression, shearing, friction, and the like.

Preliminary pulverization treatment may be performed with a device which may be used for the pulverization treatment. Specific examples of the device include a coarse crusher, such as a shredder, a jaw crusher, a gyratory crusher, a cutter mill, a cone crusher, a hammer crusher, a roll crusher, or a roll mill; or a medium crusher, such as a stamp mill, an edge runner, a cutting/shearing mill, a rod mill, an autogenous mill, or a roller mill. Of those, a cutter mill is preferred from the viewpoints of a treatment amount and a pulverization range. The time for treating the raw material is not particularly limited as long as the raw material can be homogeneously and finely pulverized by the treatment.

The grain size of the raw material subjected to pulverization in advance before its feeding is preferably a size passing through a screen (sieve) having a screen diameter of from 0.5 mm to 30 mm because, when the discharge screen diameter of the pulverization device is excessively large, the grain size of the cellulose-containing biomass increases, resulting in high sugar production cost owing to a reduction in subsequent pretreatment effects, and when the screen diameter is excessively small, pulverization cost increases. The grain size is more preferably a size passing through a screen of from 1 mm to 30 mm, most preferably a size passing through a screen of from 3 mm to 30 mm. In addition, also in the case of performing pulverization without using a screen, it is preferred to pulverize the raw material so as to achieve a size corresponding to that of a pulverized product in the case of using the screen.

[Adjustment of Water Content Ratio]

In the treatment method of the present invention, the cellulose-containing biomass serving as a raw material may be subjected to adjustment of a water content ratio in advance before its feeding to the screw extruder. As a method of adjusting a water content ratio, there are given addition of water, dewatering, and drying, in accordance with the water content ratio of the raw material before the adjustment. As described above, the water content ratio of the raw material is preferably adjusted to from 30 mass % to 80 mass % in order to optimally perform the hydrothermal reaction and achieve an optimal sealing property.

A biomass composition for saccharification can be efficiently produced by treating the biomass by the above-mentioned method. Further, when the biomass composition for saccharification produced by the above-mentioned method is hydrolyzed, a sugar can be efficiently produced. In order to efficiently produce the above-mentioned biomass composition for saccharification and sugar, a treated product after the hydrothermal treatment has an average grain size falling within a range of preferably 100 μm or less, more preferably 80 μm or less, still more preferably 60 μm or less.

### EXAMPLES

The present invention is hereinafter described by way of Examples and Comparative Examples. However, the present invention is by no means limited to the descriptions of Examples and Comparative Examples.

In Examples and Comparative Examples, screw extruders under five kinds of device conditions and a 300 mL static autoclave without a stirrer were used, and cellulose-containing biomass was treated with changing the conditions of a hydrothermal temperature and a hydrothermal time. Further, a treated sample was evaluated for a saccharification rate, and a method and conditions excellent in overall performance and efficiency including even a saccharification step were examined.

[Screw Extruder A]

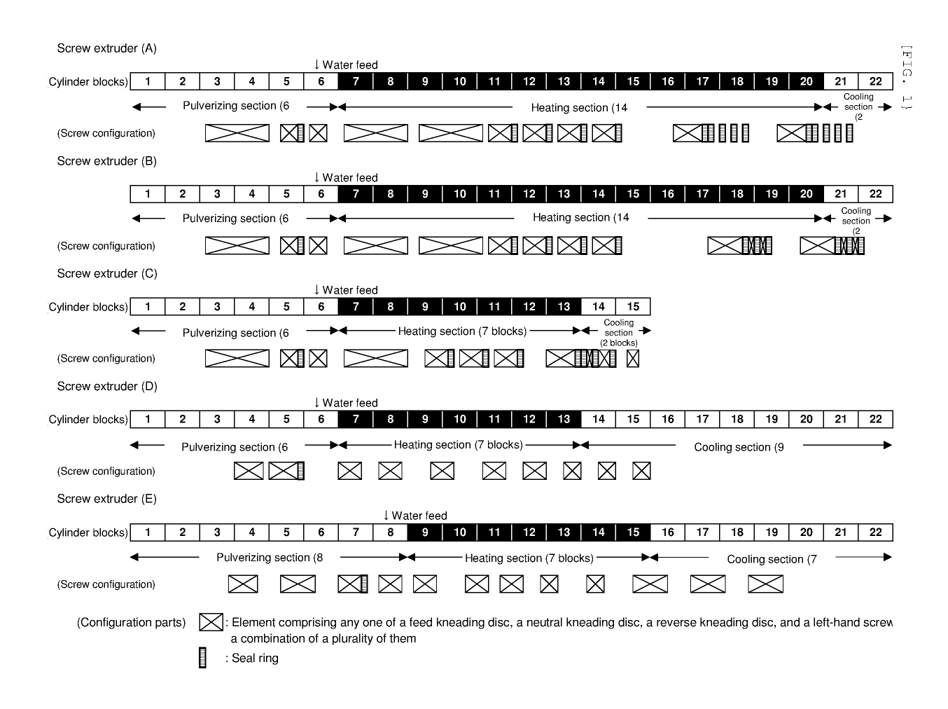

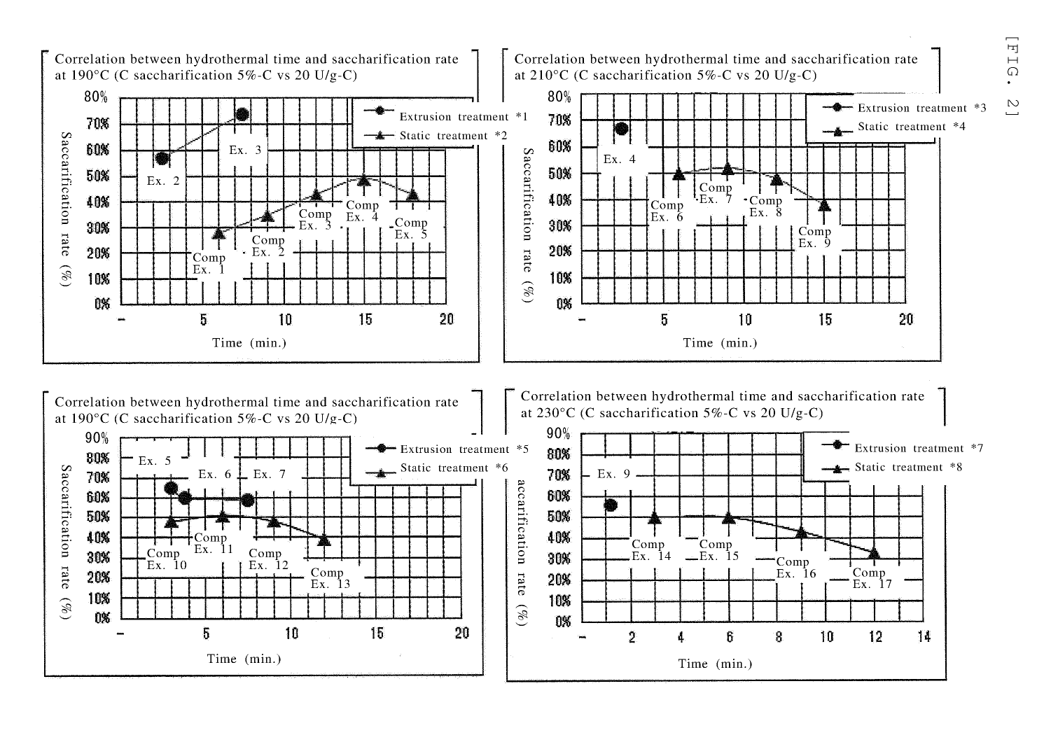

A screw extruder A in which a twin-screw extruder having an L/D of 77.0 and a screw diameter of 32 mm (trade name: TEX30α, manufactured by The Japan Steel Works, Ltd.) was allowed to have the following screw configuration illustrated in FIG. 1(A): a pulverization section in 6 blocks, a heating section in 14 blocks (6 sets of seal ring elements), and a cooling section in 2 blocks was used in Examples 3, and 5 to 7.

[Screw Extruders B to E]

Screw extruders B to E (illustrated in FIG. 1(B) to FIG. 1(E), respectively) each having the same configuration as that of the screw extruder A except that the number of blocks of the pulverization section, the number of blocks of the heating section and the number of seal ring elements in the heating section, and the number of blocks of the cooling section were changed as shown in Table 1 were used in Examples and Comparative Examples shown in Table 1.

It should be noted that an L/D value, the number of blocks of sections other than the heating section, the number of seal ring elements in these sections, the number of seal rings used, and a clearance between a seal ring and a screw were also shown in Table 1. In addition, an extruder having a cylinder diameter of 47 mm (trade name: TEX44α, manufactured by The Japan Steel Works, Ltd.) was used as the screw extruder C.

TABLE 1

| | | | | | | | | | Number of seal rings | | | |
|---|---|---|---|---|---|---|---|---|---|---|---|---|
| | L/D | | | | Number of blocks | | | | used | | | |
| Extruder | Whole | Pulverization | Heating | Cooling | Pulverization | Heating | Cooling | | Pulverization | Heating | Cooling | |
| No. | device | section | section | section | section | section | section | Total | section | section | section | Total |
| Screw | 77 | 21 | 49 | 7 | 6 | 14 | 2 | 22 | 1 | 11 | 3 | 15 |
| extruder A | | | | | | | | | | | | |
| Screw | 77 | 21 | 49 | 7 | 6 | 14 | 2 | 22 | 1 | 7 | 3 | 11 |
| extruder B | | | | | | | | | | | | |
| Screw | 52.5 | 21 | 24.5 | 7 | 6 | 7 | 2 | 15 | 1 | 4 | 3 | 8 |
| extruder C | | | | | | | | | | | | |
| Screw | 77 | 21 | 24.5 | 31.5 | 6 | 7 | 9 | 22 | 1 | 0 | 0 | 1 |
| extruder D | | | | | | | | | | | | |
| Screw | 77 | 28 | 24.5 | 24.5 | 8 | 7 | 7 | 22 | 1 | 0 | 0 | 1 |
| extruder E | | | | | | | | | | | | |

| | | | | | Clearance | | | |
|---|---|---|---|---|---|---|---|---|
| | Number of seal ring | | | | between seal | | Diameter | |
| | elements | | | | ring and screw | | of | Examples or |
| Extruder | Pulverization | Heating | Cooling | | Length | | cylinder | Comparative |
| No. | section | section | section | Total | (mm) | Ratio (%) | (mm) | Examples used |
| Screw | 1 | 6 | 0 | 7 | 0.5 | 1.6 | φ32 | Examples 3, |
| extruder A | | | | | | | | 5, 6, and 7 |
| Screw | 1 | 5 | 1 | 7 | 0.5 | 1.6 | φ32 | Example 1 |
| extruder B | | | | | | | | |
| Screw | 1 | 4 | 3 | 8 | 2.0 | 4.3 | φ47 | Examples 2, |
| extruder C | | | | | | | | 4, 8, and 9 |
| Screw | 1 | 0 | 0 | 1 | 0.5 | 1.6 | φ32 | Comparative |
| extruder D | | | | | | | | Example 28 |
| Screw | 1 | 0 | 0 | 1 | 0.5 | 1.6 | φ32 | Comparative |
| extruder E | | | | | | | | Example 17 |

[Preparation of Raw Material Bagasse]

Bagasse was used as the cellulose-containing biomass serving as a raw material.

Bagasse subjected to no treatment (water content ratio: 50%, content ratio of cellulose: 42.0%, hereinafter abbreviated as “untreated bagasse”), and bagasse (water content ratio: 10.0%, content ratio of cellulose: 38.0%, hereinafter abbreviated as “3-mm bagasse”) prepared by pulverizing air-dried bagasse with a cutter mill having a screen diameter of 3 mm (MKCM-3, manufactured by Masuko Sangyo Co., Ltd.) were used as the bagasse.

[Analysis Method for Content Ratios of Main Components of Biomass]

The content ratio of cellulose, the content ratio of hemicellulose, and the total content ratio of lignin and an ash content in the biomass were determined by an analysis method (Technical Report NREL/TP-510-42618) of The National Renewable Energy Laboratory (NREL).

[Average Grain Size]

As the average grain size of a sample subjected to hydrothermal treatment, a median diameter (cumulative median diameter) measured with a laser diffraction particle size analyzer (Microtrac MT3300 EXII, manufactured by Nikkiso Co., Ltd.) through dispersion of the sample in water was used.

[High-performance Liquid Chromatography Analysis Method and Calculation Method for Content Ratio of Cellulose]

A guard column (KS-G manufactured by Showa Denko K.K.) and a separation column (KS-802 manufactured by Showa Denko K.K.) were connected to each other, and the column temperature was set to 75° C. Pure water was supplied as an eluting solution at a rate of 0.5 ml/min, and a separated component was subjected to quantitative determination with a differential refractive index detector. Thus, the concentration of glucose was determined, and the content ratio of cellulose was calculated based on the following equation.

Content ratio of cellulose (%)={mass of filtrate (g)×(concentration of glucose (%)/100)×0.9}/mass of weighed biomass (g)×100

(The numerical value “0.9” in the equation is a coefficient for correcting changes in molecular weight caused by hydrolysis of cellulose.)

[Calculation Method for Recovery Rate of Cellulose in Pretreatment]

The recovery rate of cellulose from raw material biomass in pretreatment was calculated based on the following equation.

Recovery rate of cellulose (%)={dry mass of hydrothermally treated product (g) after washing with water/dry mass of raw material biomass (g)}×{content ratio of cellulose (%) in hydrothermally treated product/content ratio of cellulose (%) in raw material biomass}×100

[Measurement of Saccharification Performance with Enzyme]

Preparation of Acid Buffer Solution:

30 g of acetic acid was put in a 100 ml measuring flask, and diluted with pure water to give a 3 M acetic acid aqueous solution. 41 g of sodium acetate was put in a 100 ml measuring flask, and diluted with pure water to give a 3 M sodium acetate aqueous solution. The 5 M acetic acid aqueous solution was added to the 3 M sodium acetate aqueous solution until the pH became 5.0. Thus, an acetic acid buffer solution was obtained.

Preparation of Enzyme Solution:

1.5 g of Meicelase (trademark, cellulase manufactured by Meiji Seika Kaisha, Ltd. (currently Meiji Seika Pharma Co., Ltd.)) was dissolved in 98.5 g of pure water.

The FPU activity (Filter Paper Assay for Saccharifying Cellulase) of the enzyme solution was 6 FPU/g, which was determined according to an analysis method of International Union of Pure and Applied Chemistry (IUPAC) (Pure & Appl. Chem., Vol. 59, No. 2, pp. 257-268, 1987).

Saccharification Reaction:

A rotor was put in a 50 ml glass vessel with a cover, and a composition subjected to pretreatment was weighed so that the amount of cellulose was 0.5 g. Then, 0.6 g of the acetic acid buffer solution and 1.03 g of the enzyme solution were added thereto, and further, pure water was added thereto to give a total of 10 g. The resultant was subjected to a saccharification reaction with the enzyme in a thermostat bath at 40° C. for 72 hours (Hr) while being stirred. The resultant saccharified solution was subjected to quantitative determination for glucose by high-performance liquid chromatography analysis. Thus, a saccharification rate and a sugar utilization rate were calculated by the following equations.

Saccharification rate (%)={concentration of glucose (%) in reaction solution×0.9}/concentration of cellulose (%) in reaction solution at the beginning of reaction}×100

(The numerical value “0.9” in the equation is a coefficient for correcting changes in molecular weight caused by hydrolysis of cellulose.)

Sugar utilization rate (%)=recovery rate of cellulose (%)×saccharification rate (%)/100

### Example 1

The 3-mm bagasse was loaded into the screw extruder B having a screw rotation number of 350 rpm with a mass feeder and a compactor at a feed rate of 5.0 kg/Hr of a mass of the bagasse as it was and 4.5 kg/Hr in terms of dry mass. In the pulverization section (6 blocks in a cylinder), water was added through a liquid feed line at a feed rate of 4.8 kg/Hr, and a water content ratio was adjusted so that a water content ratio of 54 mass % (solid content concentration: 46 mass %) was continuously obtained in the pulverization section upstream of the heating section. The raw material was subjected to hydrothermal treatment in the heating section so that the temperature of the raw material in the heating section (hereinafter abbreviated as “hydrothermal temperature”) and a pressure in the heating section were set to 175° C. and 5 MPa, respectively, and then cooled to 70° C. or less in the cooling section with a water cooling jacket, followed by recovery of a sample from a discharge port. A time of passage through the heating section (hereinafter abbreviated as “hydrothermal time”) under such conditions was 7.5 minutes. The treated sample recovered had an average grain size of 32 μm. 138 g of water, which was three times the amount of a solid content in the sample, was added to 100 g of the treated sample recovered, and the resultant was suspended, followed by centrifugal filtration with a centrifugal filter (H-122, manufactured by Kokusan Co., Ltd., filter cloth: cotton) at 3,000 rpm. Thus, a water-containing solid content was obtained. The obtained water-containing solid content was calculated for a recovery rate of cellulose, a saccharification rate, and a sugar utilization rate by the above-mentioned methods.

### Examples 2 to 9 and Comparative Examples 27 and 28

The recovery rate of cellulose, saccharification rate, and sugar utilization rate were calculated in the same manner as in Example 1 except that the conditions were changed as shown in Table 2.

### Comparative Examples 1 to 17

16.7 g of the 3-mm bagasse and 133.3 g of pure water were loaded into a 300-mL autoclave without a stirrer (High-Pressure Microreactor MMJ-300, manufactured by OM Lab-Tech Co., Ltd,) so that the total mass of a slurry was 150 g and a water content ratio was 90% (solid content concentration: 10%), and the autoclave was sealed. After that, the slurry was heated to a hydrothermal temperature shown in Comparative Examples 1 to 17 of Table 3 with a heater and retained at the temperature for a hydrothermal time shown in Table 3. Immediately, cooling was performed by removing the autoclave and immersing the autoclave in water put in a bucket. After the cooling, the entire amount of the slurry was recovered from the autoclave, and subjected to centrifugal filtration with a centrifugal filter (H-122, manufactured by Kokusan Co., Ltd., filter cloth: cotton) at 3,000 rpm. Thus, water-containing solid contents were obtained. All of them had an average grain size of 500 82 m or more through microscopic observation. The obtained water-containing solid contents were each calculated for a recovery rate of cellulose, a saccharification rate, and a sugar utilization rate by the above-mentioned methods.

### Comparative Examples 18 to 26

The recovery rate of cellulose, saccharification rate, and sugar utilization rate were calculated in the same manner as in Comparative Example 1 except that the conditions were changed as shown in Table 3.

For treated products of biomass recovered, the results of the average grain size, recovery rate of cellulose, saccharification rate, and sugar utilization rate were shown in Table 2 for Examples 1 to 9 and Comparative Examples 27 and 29, and in Table 3 for Comparative Examples 1 to 26.

TABLE 2

| | | | | | | | Water content | |
|---|---|---|---|---|---|---|---|---|
| | | | | | | | ratio in | |
| | | Feed rate | | | | Water feed rate | pulverization | |
| | | | In terms | Screw | | through | section | Hydro- |
| | | Mass | of dry | rotation | | liquid feed | (solid content | thermal |
| | Kind of | as it is | mass | number | | line | concentration) | temperature |
| | bagasse | [kg/Hr] | [kg/Hr] | [rpm] | Screw extruder | [kg/Hr] | [mass %] | [° C.] |
| Example 1 | 3-mm | 5.0 | 4.5 | 350 | Hydro-thermal | 4.8 | 54 (46) | 175 |
| | bagasse | | | | extruder B | | | |
| Example 2 | Untreated | 20.0 | 10.0 | 200 | Hydro-thermal | 5.0 | 60 (40) | 190 |
| | bagasse | | | | extruder C | | | |
| Example 3 | 3-mm | 5.0 | 4.5 | 350 | Hydro-thermal | 3.6 | 48 (52) | 190 |
| | bagasse | | | | extruder A | | | |
| Example 4 | Untreated | 20.0 | 10.0 | 200 | Hydro-thermal | 5.0 | 60 (40) | 210 |
| | bagasse | | | | extruder C | | | |
| Example 5 | 3-mm | 12.5 | 11.3 | 350 | Hydrothermal | 5.8 | 39 (61) | 220 |
| | bagasse | | | | extruder A | | | |
| Example 6 | 3-mm | 10.0 | 9.0 | 350 | Hydrothermal | 4.6 | 38 (62) | 220 |
| | bagasse | | | | extruder A | | | |
| Example 7 | 3-mm | 5.0 | 4.5 | 350 | Hydrothermal | 3.6 | 48 (52) | 220 |
| | bagasse | | | | extruder A | | | |
| Example 8 | Untreated | 20.0 | 10.0 | 100 | Hydrothermal | 5.0 | 60 (40) | 230 |
| | bagasse | | | | extruder C | | | |
| Example 9 | Untreated | 40.0 | 20.0 | 200 | Hydrothermal | 10.0 | 60 (40) | 230 |
| | bagasse | | | | extruder C | | | |
| Comparative | 3-mm | 2.5 | 2.3 | 350 | Hydrothermal | 2.9 | 58 (43) | 175 |
| Example 27 | bagasse | | | | extruder E | | | |
| Comparative | 3-mm | 2.5 | 2.3 | 350 | Hydrothermal | 2.9 | 58 (43) | 190 |
| Example 28 | bagasse | | | | extruder D | | | |

| | | | Maximum | | | | | |
|---|---|---|---|---|---|---|---|---|
| | | | grain size in | | | | | |
| | | | pulverization | | Water in an | | | |
| | | | section | Average | amount three | | | |
| | | | immediately | grain | times the | | | |
| | | Hydro- | upstream of | size after | amount of | Recovery | | Sugar |
| | | thermal | heating | hydrothermal | solid content | rate of | Saccharification | utilization |
| | Pressure | time | section | treatment | in sample | cellulose | rate | rate |
| | [MPa] | [min] | [μm] | [μm] | [g] | [%] | [%] | [%] |
| Example 1 | 5 | 7.5 | <1,000 | 32 | 138 | 96 | 66 | 63 |
| Example 2 | 5 | 2.5 | <1,000 | 15 | 120 | 93 | 57 | 53 |
| Example 3 | 5 | 7.5 | <1,000 | 27 | 156 | 92 | 74 | 68 |
| Example 4 | 5 | 2.5 | <1,000 | 12 | 120 | 93 | 67 | 62 |
| Example 5 | 5 | 3.0 | <1,000 | 4 | 183 | 91 | 65 | 59 |
| Example 6 | 5 | 3.8 | <1,000 | 4 | 186 | 92 | 60 | 55 |
| Example 7 | 5 | 7.5 | <1,000 | 3 | 156 | 87 | 59 | 51 |
| Example 8 | 5 | 2.5 | <1,000 | 47 | 100 | 92 | 52 | 48 |
| Example 9 | 5 | 1.2 | <1,000 | 52 | 100 | 95 | 56 | 53 |
| Comparative | 5 | 7.5 | <1,000 | 127 | 126 | 9 | 30 | 29 |
| Example 27 | | | | | | | | |
| Comparative | 5 | 7.5 | <1,000 | 131 | 126 | 93 | 34 | 32 |
| Example 28 | | | | | | | | |

TABLE 3

| | | Water | | | | | | |
|---|---|---|---|---|---|---|---|---|
| | | content | | | Mass | | | |
| | Total | ratio | | | of | | | |
| | mass | (solid | | | pure | | Hydro- | Hydro- |
| | of | content | | Mass of | water | | thermal | thermal |
| | slurry | concentration) | Kind of | bagasse | added | Hydrothermal | temperature | time |
| | [g] | [%] | bagasse | [g] | [g] | method | [° C.] | [min] |
| Comparative | 150 | 90 (10) | 3-mm | 16.7 | 100.0 | Static | 190 | 6.0 |
| Example 1 | | | bagasse | | | hydrothermal | | |
| | | | | | | treatment | | |
| Comparative | 150 | 90 (10) | 3-mm | 16.7 | 100.0 | Static | 190 | 9.0 |
| Example 2 | | | bagasse | | | hydrothermal | | |
| | | | | | | treatment | | |
| Comparative | 150 | 90 (10) | 3-mm | 16.7 | 100.0 | Static | 190 | 12.0 |
| Example 3 | | | bagasse | | | hydrothermal | | |
| | | | | | | treatment | | |
| Comparative | 150 | 90 (10) | 3-mm | 16.7 | 100.0 | Static | 190 | 15.0 |
| Example 4 | | | bagasse | | | hydrothermal | | |
| | | | | | | treatment | | |
| Comparative | 150 | 90 (10) | 3-mm | 16.7 | 100.0 | Static | 190 | 18.0 |
| Example 5 | | | bagasse | | | hydrothermal | | |
| | | | | | | treatment | | |
| Comparative | 150 | 90 (10) | 3-mm | 16.7 | 100.0 | Static | 210 | 6.0 |
| Example 6 | | | bagasse | | | hydrothermal | | |
| | | | | | | treatment | | |
| Comparative | 150 | 90 (10) | 3-mm | 16.7 | 100.0 | Static | 210 | 9.0 |
| Example 7 | | | bagasse | | | hydrothermal | | |
| | | | | | | treatment | | |
| Comparative | 150 | 90 (10) | 3-mm | 16.7 | 100.0 | Static | 210 | 12.0 |
| Example 8 | | | bagasse | | | hydrothermal | | |
| | | | | | | treatment | | |
| Comparative | 150 | 90 (10) | 3-mm | 16.7 | 100.0 | Static | 210 | 15.0 |
| Example 9 | | | bagasse | | | hydrothermal | | |
| | | | | | | treatment | | |
| Comparative | 150 | 90 (10) | 3-mm | 16.7 | 100.0 | Static | 220 | 3.0 |
| Example 10 | | | bagasse | | | hydrothermal | | |
| | | | | | | treatment | | |
| Comparative | 150 | 90 (10) | 3-mm | 16.7 | 100.0 | Static | 220 | 6.0 |
| Example 11 | | | bagasse | | | hydrothermal | | |
| | | | | | | treatment | | |
| Comparative | 150 | 90 (10) | 3-mm | 16.7 | 100.0 | Static | 220 | 9.0 |
| Example 12 | | | bagasse | | | hydrothermal | | |
| | | | | | | treatment | | |
| Comparative | 150 | 90 (10) | 3-mm | 16.7 | 100.0 | Static | 220 | 12.0 |
| Example 13 | | | bagasse | | | hydrothermal | | |
| | | | | | | treatment | | |
| Comparative | 150 | 90 (10) | 3-mm | 16.7 | 100.0 | Static | 230 | 3.0 |
| Example 14 | | | bagasse | | | hydrothermal | | |
| | | | | | | treatment | | |
| Comparative | 150 | 90 (10) | 3-mm | 16.7 | 100.0 | Static | 230 | 6.0 |
| Example 15 | | | bagasse | | | hydrothermal | | |
| | | | | | | treatment | | |
| Comparative | 150 | 90 (10) | 3-mm | 16.7 | 100.0 | Static | 230 | 9.0 |
| Example 16 | | | bagasse | | | hydrothermal | | |
| | | | | | | treatment | | |
| Comparative | 150 | 90 (10) | 3-mm | 16.7 | 100.0 | Static | 230 | 12.0 |
| Example 17 | | | bagasse | | | hydrothermal | | |
| | | | | | | treatment | | |
| Comparative | 150 | 70 (30) | 3-mm | 50.0 | 100.0 | Static | 190 | 15.0 |
| Example 18 | | | bagasse | | | hydrothermal | | |
| | | | | | | treatment | | |
| Comparative | 150 | 50 (50) | 3-mm | 83.3 | 66.7 | Static | 190 | 15.0 |
| Example 19 | | | bagasse | | | hydrothermal | | |
| | | | | | | treatment | | |
| Comparative | 150 | 70 (30) | 3-mm | 116.7 | 33.3 | Static | 190 | 15.0 |
| Example 20 | | | bagasse | | | hydrothermal | | |
| | | | | | | treatment | | |
| Comparative | 150 | 70 (30) | 3-mm | 50.0 | 100.0 | Static | 210 | 9.0 |
| Example 21 | | | bagasse | | | hydrothermal | | |
| | | | | | | treatment | | |
| Comparative | 150 | 50 (50) | 3-mm | 83.3 | 66.7 | Static | 210 | 9.0 |
| Example 22 | | | bagasse | | | hydrothermal | | |
| | | | | | | treatment | | |
| Comparative | 150 | 70 (30) | 3-mm | 116.7 | 33.3 | Static | 210 | 9.0 |
| Example 23 | | | bagasse | | | hydrothermal | | |
| | | | | | | treatment | | |
| Comparative | 150 | 70 (30) | 3-mm | 50.0 | 100.0 | Static | 220 | 6.0 |
| Example 24 | | | bagasse | | | hydrothermal | | |
| | | | | | | treatment | | |
| Comparative | 150 | 50 (50) | 3-mm | 83.3 | 66.7 | Static | 220 | 6.0 |
| Example 25 | | | bagasse | | | hydrothermal | | |
| | | | | | | treatment | | |
| Comparative | 150 | 70 (30) | 3-mm | 116.7 | 33.3 | Static | 220 | 6.0 |
| Example 26 | | | bagasse | | | hydrothermal | | |
| | | | | | | treatment | | |

| | Maximum grain | | | | | |
|---|---|---|---|---|---|---|
| | size in | | Water in an | | | |
| | pulverization | | amount | | | |
| | section | Average | three times | | | |
| | immediately | grain size | the amount | | | |
| | upstream of | after | of solid | Recovery | | Sugar |
| | heating | hydrothermal | content in | rate of | Saccharification | utilization |
| | section | treatment | sample | cellulose | rate | rate |
| | [μm] | [μm] | [g] | [%] | [%] | [%] |
| Comparative | <3,000 (*1) | >500 (*2) | 45 | 91 | 28 | 25 |
| Example 1 | | | | | | |
| Comparative | <3,000 (*1) | >500 (*2) | 45 | 90 | 35 | 32 |
| Example 2 | | | | | | |
| Comparative | <3,000 (*1) | >500 (*2) | 45 | 89 | 43 | 38 |
| Example 3 | | | | | | |
| Comparative | <3,000 (*1) | >500 (*2) | 45 | 88 | 49 | 43 |
| Example 4 | | | | | | |
| Comparative | <3,000 (*1) | >500 (*2) | 45 | 86 | 43 | 37 |
| Example 5 | | | | | | |
| Comparative | <3,000 (*1) | >500 (*2) | 45 | 90 | 50 | 45 |
| Example 6 | | | | | | |
| Comparative | <3,000 (*1) | >500 (*2) | 45 | 89 | 52 | 46 |
| Example 7 | | | | | | |
| Comparative | <3,000 (*1) | >500 (*2) | 45 | 88 | 48 | 42 |
| Example 8 | | | | | | |
| Comparative | <3,000 (*1) | >500 (*2) | 45 | 86 | 38 | 33 |
| Example 9 | | | | | | |
| Comparative | <3,000 (*1) | >500 (*2) | 45 | 91 | 48 | 44 |
| Example 10 | | | | | | |
| Comparative | <3,000 (*1) | >500 (*2) | 45 | 90 | 51 | 46 |
| Example 11 | | | | | | |
| Comparative | <3,000 (*1) | >500 (*2) | 45 | 88 | 48 | 42 |
| Example 12 | | | | | | |
| Comparative | <3,000 (*1) | >500 (*2) | 45 | 85 | 39 | 33 |
| Example 13 | | | | | | |
| Comparative | <3,000 (*1) | >500 (*2) | 45 | 89 | 50 | 45 |
| Example 14 | | | | | | |
| Comparative | <3,000 (*1) | >500 (*2) | 45 | 88 | 50 | 44 |
| Example 15 | | | | | | |
| Comparative | <3,000 (*1) | >500 (*2) | 45 | 85 | 43 | 37 |
| Example 16 | | | | | | |
| Comparative | <3,000 (*1) | >500 (*2) | 45 | 82 | 33 | 27 |
| Example 17 | | | | | | |
| Comparative | <3,000 (*1) | >500 (*2) | 135 | 88 | 48 | 42 |
| Example 18 | | | | | | |
| Comparative | <3,000 (*1) | >500 (*2) | 225 | 88 | 42 | 37 |
| Example 19 | | | | | | |
| Comparative | <3,000 (*1) | >500 (*2) | 315 | 90 | 31 | 28 |
| Example 20 | | | | | | |
| Comparative | <3,000 (*1) | >500 (*2) | 135 | 89 | 51 | 45 |
| Example 21 | | | | | | |
| Comparative | <3,000 (*1) | >500 (*2) | 225 | 90 | 43 | 39 |
| Example 22 | | | | | | |
| Comparative | <3,000 (*1) | >500 (*2) | 315 | 91 | 33 | 30 |
| Example 23 | | | | | | |
| Comparative | <3,000 (*1) | >500 (*2) | 135 | 90 | 51 | 46 |
| Example 24 | | | | | | |
| Comparative | <3,000 (*1) | >500 (*2) | 225 | 91 | 45 | 41 |
| Example 25 | | | | | | |
| Comparative | <3,000 (*1) | >500 (*2) | 315 | 91 | 32 | 29 |
| Example 26 | | | | | | |

(*1) Passing through 3-mm screen
(*2) Approximate value through microscopic observation

[Saccharification Performance in Static Hydrothermal Treatment]

From the results of hydrothermal treatment at a solid content concentration of 10 mass % in each of Comparative Examples 1 to 17, the tendencies of the following rates with respect to a hydrothermal time were confirmed at each hydrothermal temperature: a saccharification rate of a hydrothermally treated product to glucose; a recovery rate of cellulose in the hydrothermal treatment; and a sugar utilization rate, which was an indicator of overall efficiency from the hydrothermal treatment to saccharification.

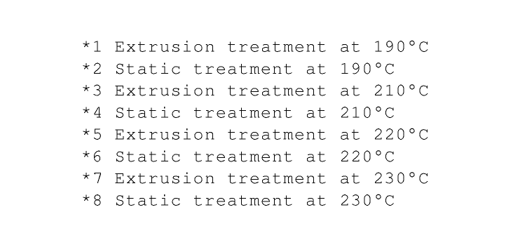

The correlation between a hydrothermal time and a saccharification rate at each hydrothermal temperature is shown in FIG. 2.

At each temperature, the saccharification rate had a tendency to have an optimal hydrothermal time range. It is presumed that, when the hydrothermal time is shorter than a hydrothermal time in the optimal range, the action of the pretreatment on the biomass is weak, resulting in a reduction in saccharification rate, and when the hydrothermal time is longer than the hydrothermal time in the optimal range, impurities are altered or an excessively decomposed product of a sugar and the like increase, resulting in a reduction in saccharification rate. The saccharification rate had the following maximum values at 190° C., 210° C., 220° C., and 230° C., respectively: 49% in a hydrothermal time of 15 minutes (Comparative Example 4), 52% in 9 minutes (Comparative Example 7), 51% in 6 minutes (Comparative Example 11), and 50% in 3 minutes and 6 minutes (Comparative Examples 14 and 15).

The recovery rate of cellulose had a tendency to decrease with an increase in retention time at each temperature, and had a tendency to decrease with an increase in hydrothermal temperature in each retention time. However, the recovery rate of cellulose was around 90% around retention times in which the saccharification rate had the maximum values, and thus had less influence on the tendency of the sugar utilization rate for optimal conditions, the sugar utilization rate being calculated by multiplication of the recovery rate of cellulose by a saccharification rate.

From Table 3, it is revealed that, at each hydrothermal temperature, the correlation between a hydrothermal time and a saccharification rate is similar to the correlation between a hydrothermal time and a saccharification rate. The sugar utilization rate had the following maximum values at 190° C., 210° C., 220° C., and 230° C., respectively: 43% in a hydrothermal time of 15 minutes (Comparative Example 4), 46% in 9 minutes (Comparative Example 7), 46% in 6 minutes (Comparative Example 11), and 45% in 3 minutes (Comparative Example 14).

[Saccharification Performance in Extrusion Hydrothermal Treatment]

From FIG. 2, it was confirmed that, at each temperature, the saccharification rate in extrusion hydrothermal treatment (Examples 1 to 9), which was hydrothermal treatment with a screw extruder, exceeded the maximum value in the static hydrothermal treatment in a time shorter than the optimal hydrothermal time in the static hydrothermal treatment, as compared to the results of the static hydrothermal treatment (Comparative Examples 1 to 17) at the same hydrothermal temperature.

The recovery rate of cellulose exceeded 90% in each Example, which was comparable to or higher than the values in the static hydrothermal treatment. In addition, as in the static hydrothermal treatment, the recovery rate of cellulose had less influence on the tendency of the sugar utilization rate for optimal conditions.

From Table 2, it is revealed that, in the extrusion hydrothermal treatment, the correlation between a hydrothermal time and a sugar utilization rate is similar to the correlation between a hydrothermal time and a saccharification rate at each temperature. It was confirmed that, at each temperature, a sugar utilization rate higher than the maximum value in the static hydrothermal treatment was obtained in a shorter hydrothermal time. Thus, it was confirmed that the extrusion hydrothermal treatment had an effect of improving productivity per unit time by virtue of a reduction in reaction time.

It is presumed that such tendencies in the extrusion hydrothermal treatment are obtained by a synergistic effect of the hydrothermal treatment and kneading stress applied during the hydrothermal treatment.

The maximum value of the sugar utilization rate in the extrusion hydrothermal treatment in Examples 1 to 9 was 68% at 190° C. in 7.5 minutes (Example 3), and the second maximum value was 63% at 175° C. in 7.5 minutes. It is presumed that, with regard to the relationship between the hydrothermal conditions (temperature and time) and the saccharification performance in the extrusion hydrothermal treatment, the saccharification performance has a tendency to have an optimal treatment time range at each temperature as in the static hydrothermal treatment when other conditions including device conditions and a solid content concentration are the same. From Tables 2 and 3 and FIG. 2, it is presumed that, under the conditions in which a kneading force is sufficient at the time of heating, a heating time range exhibiting superior saccharification performance as compared to the static hydrothermal treatment is 15 minutes or less at 190° C., and 10 minutes or less at 210° C., 220° C., and 230° C., and a range exhibiting optimal performance is 10 minutes or less at 190° C., and 5 minutes or less at 210° C., 220° C., and 230° C.

[Hydrothermal Treatment at High Solid Content Concentration]

The solid content concentrations at the time of hydrothermal treatment in the extrusion hydrothermal treatment

(Examples 1 to 9 and Comparative Examples 27 and 28) were from 40 mass % to 62 mass % as shown in Table 2. Treatment of a raw material slurry in a solid form having a high concentration and low flowability was realized. This results from a high extrusion function exhibited by a screw, which is a feature of the screw extruder. In Comparative Examples 18 to 26, in order to confirm the saccharification performance in the static hydrothermal treatment in a range of the solid content concentrations in the extrusion hydrothermal treatment, the hydrothermal treatment was performed under the conditions of 190° C. and 15 minutes, 210° C. and 9.0 minutes, and 220° C. and 6 minutes, which were the optimal conditions at the respective temperatures in the static hydrothermal treatment at a solid content concentration of 10 mass %, by increasing the solid content concentration to 30 mass %, 50 mass %, and 70 mass % (Tables 2 and 3).

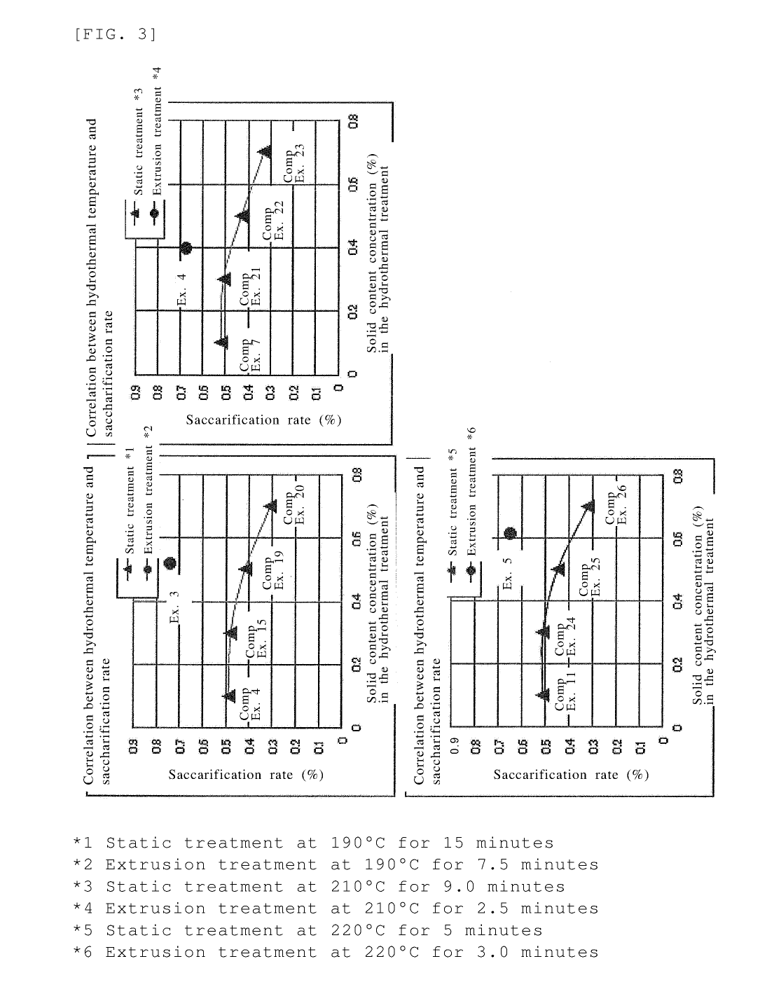

As shown in FIG. 3, under the respective temperature conditions, the saccharification rate decreased with an increase in solid content concentration. In addition, the recovery rate of cellulose had a tendency to increase with an increase in solid content concentration. However, all the recovery rates of cellulose were around 90%, and hence the increase in recovery rate of cellulose was not so much as to have an influence on the tendency of the sugar utilization rate.

Under the respective temperature conditions, the correlation between a sugar utilization rate and a solid content concentration is similar to the correlation between a saccharification rate and a solid content concentration. When the static hydrothermal treatment was compared to the extrusion hydrothermal treatment in a comparable solid content concentration range, a difference in the sugar utilization rate was further increased, and it was confirmed that the extrusion hydrothermal treatment had superior efficiency. It can be said that another feature of the extrusion hydrothermal treatment is that the treatment can be performed at a high solid content concentration.

[Maximum Grain Size before Hydrothermal Treatment]

With regard to the grain sizes of the samples subjected to the extrusion hydrothermal treatment and the static hydrothermal treatment in Examples and Comparative Examples, the maximum grain sizes before the hydrothermal treatment and the average grain sizes after the hydrothermal treatment were shown in Table 2 for Examples 1 to 9 and Comparative Examples 27 and 28 and in Table 3 for Comparative Examples 1 to 26.

The maximum grain sizes before the static hydrothermal treatment were each 3,000 μm or less because of the use of the 3-mm bagasse pulverized with a cutter mill having a screen diameter of 3 mm.

The maximum grain sizes before the extrusion hydrothermal treatment were each determined through microscopic observation of the sample extracted from the pulverization section immediately upstream of the heating section. The maximum grain sizes of the samples were each 1,000 μm or less because of coarse pulverization in the pulverization section in the device upstream of the heating section in the extrusion hydrothermal treatment.

In addition, the 3-mm bagasse was tried to be treated in a screw extruder having a screw configuration with a low kneading force by removing the seal ring and reducing the number of kneading discs in each of the pulverization sections of the screw extruders A and E, but the pressure of vapor was not able to be maintained on an upstream side, and a backward flow was generated. The treatment failed. In those cases, the course pulverization of the raw material in the pulverization section before the hydrothermal treatment was weak, and the maximum grain sizes each exceeded 1,000 μm.

From the above-mentioned results, it is presumed that, in the extrusion hydrothermal treatment, the course pulverization in the pulverization section exhibits an effect of improving the saccharification performance and an effect of sealing vapor to be generated in the hydrothermal section on an upstream side. It was confirmed that the course pulverization was preferably performed to such degree that the maximum grain size fell below 1,000 μm.

[Average Grain Size after Hydrothermal Treatment]

The average grain sizes after the hydrothermal treatment were determined through analysis of the samples with Microtrac. The average grain sizes after the static hydrothermal treatment were each exceeded 500 μm. Possible causes for this are as follows: the raw material before the hydrothermal treatment had a large maximum grain size; and physical stress, such as stirring or pulverization, was not applied also in the hydrothermal treatment.

On the other hand, the average grain sizes after the extrusion hydrothermal treatment were 100 μm or less in Examples 1 to 8 exhibiting high saccharification performance, while the average grain sizes exceeded 100 μm in Comparative Examples 27 and 28 not exhibiting high saccharification performance.

The cause for the small average grain sizes in the extrusion hydrothermal treatment as compared to the static hydrothermal treatment is pulverization of the raw material in the pulverization section in the device before the hydrothermal treatment, and further pulverization of the raw material also in the hydrothermal treatment by a kneading force exhibited by a screw. In addition, a possible cause for the small average grain sizes in Examples 1 to 8 as compared to Comparative Examples 27 and 28 in the extrusion hydrothermal treatment is as follows: the seal ring element having a high grinding effect is included in the heating section in Examples 1 to 8, but such seal ring element is not included in the heating section in Comparative Examples 27 and 28.

From the above-mentioned results, it is suggested that a sample subjected to the hydrothermal treatment preferably has an average grain size of 100 μm or less in order to achieve high saccharification performance in the extrusion hydrothermal treatment.

[Operation Results in Terms of Conditions of Cooling Section of Screw Extruder]

Examples and Comparative Examples in this description were test examples in which the hydrothermal treatment was able to be performed with a screw extruder. In each screw extruder, a cooling system of a water cooling jacket and a liquid feed line was used in the cooling section, and a pressure regulating valve was mounted to a discharge port. In the test examples, the temperature in the cooling section was reduced to 70° C. or less through cooling with a water cooling jacket in Examples 1 to 7 and Comparative Examples 27 and 28 and through cooling with the water cooling jacket and water feeding from a liquid feed line to the biomass to be treated in combination in Examples 8 and 9. The hydrothermal treatment was able to be stably and continuously performed in all the test examples.

Meanwhile, a test was performed, but the hydrothermal treatment was not able to be actually performed under the following conditions. When the treatment was performed without using any cooling system of a water cooling jacket and a liquid feed line in each screw extruder, the temperature in the cooling section kept a high temperature state exceeding 100° C., a sample intermittently jetted together with vapor, and the treatment was not able to be stably performed in the case of each screw extruder. As described above, it was confirmed that, for stable operation, the temperature in the cooling section needed to be reduced to 70° C. or less through use of the cooling system of a water cooling jacket and/or a liquid feed line.

[Conditions of Extruder in Extrusion Hydrothermal Treatment]

The extrusion hydrothermal treatment in Examples 1 to 8 was performed with the screw extruders A to C (FIG. 1(A) to FIG. 1(C)) under the conditions in which 4 to 6 sets of elements each including a seal ring and at least one set of a kneading disc and/or a left-hand screw arranged immediately upstream of the seal ring (seal ring elements) were arranged in the heating section. In contrast, in Comparative Examples 27 and 28, the extrusion hydrothermal treatment was performed with the screw extruders D and E (FIG. 1(D) and FIG. 1(E)) under the conditions in which the seal ring element was not arranged in the heating section.

A difference in the sugar utilization rate depending on the number of seal ring elements to be included was considered under the same hydrothermal conditions. As a result, it was found that, under the hydrothermal conditions of 175° C. and 7.5 minutes, the sugar utilization rate in Example 1 including 5 sets of seal ring elements was 63%, whereas the sugar utilization rate in Comparative Example 27 including 0 sets of seal ring elements was 28%, and under the hydrothermal conditions of 190° C. and 7.5 minutes, the sugar utilization rate in Example 3 including 6 sets of seal ring elements was 68%, whereas the sugar utilization rate in Comparative Example 28 including 0 sets of seal ring elements was 32%. It was suggested that the sugar utilization rate depended on the presence or absence of the seal ring element.

Such effect exhibited by the seal ring element is presumed to be caused as follows: high grinding stress is applied to the cellulose-containing biomass serving as a raw material when it passes through an extremely narrow clearance portion between the seal ring and the cylinder concurrently with the hydrothermal treatment, and thus the saccharification performance of cellulose in the biomass is improved.

From the above-mentioned results, it was confirmed that as device conditions of a screw extruder for obtaining a cellulose-containing composition exhibiting a high sugar utilization rate, arrangement of a plurality of seal ring elements in the heating section was effective.

### INDUSTRIAL APPLICABILITY

According to the treatment method for biomass of the present invention, an industrially useful composition exhibiting high saccharification performance in saccharification of cellulose-containing biomass can be efficiently obtained at a high concentration in a short time.

1. A treatment method for biomass, which is a pretreatment method comprising continuously performing hydrothermal treatment through use of a screw extruder to produce a biomass composition for saccharification from cellulose-containing biomass serving as a raw material,
the treatment method sequentially comprising:
pulverizing, in a pulverization section of the screw extruder, the cellulose-containing biomass
serving as a raw material so as to have a maximum grain size of 1,000 μm or less and
adjusting a water content ratio thereof to from 20% to 80%;
performing, in a heating section of the screw extruder, hydrothermal treatment at a temperature of from 150° C. to 250° C. for 0.1 minute to 10 minutes while performing kneading pulverization having a grinding effect; and
cooling, in a cooling section of the screw extruder downstream of the heating section, a treated product to 100° C. or less to recover the treated product of the cellulose-containing biomass having an average grain size of 100 μm or less.

2. The treatment method for biomass as claimed in claim 1, in which, in the heating section, the kneading pulverization having a grinding effect is performed concurrently with the hydrothermal treatment by arranging an element including a seal ring and at least one set of a kneading disc and/or a left-hand screw arranged immediately upstream of the seal ring.

3. The treatment method for biomass as claimed in claim 1, in which, in the cooling section downstream of the heating section, the cooling is performed by installing a water cooling jacket and/or a liquid feed line.

4. The treatment method for biomass as claimed in claim 1, in which the screw extruder is a co-rotating twin-screw extruder.

5. The treatment method for biomass as claimed in claim 1, in which the cellulose-containing biomass is soft biomass.

6. A production method for a biomass composition for saccharification, comprising conducting the treatment method as claimed in claim 1.

7. A production method for a sugar, comprising hydrolyzing a biomass composition obtained by the production method as claimed in claim 6.
